(19) United States
(12) Patent Application Publication
(10) Pub. No.: US 2019/0030106 A1
(43) Pub. Date: Jan. 31, 2019

(54) Composition of Plant Extracts That Increase Döderlein's Bacilli, Used for the Treatment of Sexually Transmitted Diseases (STD) and the Human Papillomavirus (HPV), Having an Aphrodisiac Effect

(71) Applicant: María del Carmen Ramírez Olvera, Col. Benito Juarez (MX)

(72) Inventor: María del Carmen Ramírez Olvera, Col. Benito Juarez (MX)

(21) Appl. No.: 16/075,795

(22) PCT Filed: Oct. 11, 2016
(86) PCT No.: PCT/MX2016/000107

§ 371 Date: Aug. 6, 2018

(30) Foreign Application Priority Data
Feb. 4, 2016 (MX) MX/A/2016/001612

(51) Int. Cl. A61K 36/28; A61P 31/04; A61K 36/31; A61K 36/82; A61K 36/8962; A61K 36/9068; A61P 15/02

(52) U.S. Cl. CPC A61K 36/28; A61P 31/04; A61K 36/31; A61K 9/0014; A61K 36/8962; A61K 36/9068; A61P 15/02; A61K 36/82

(57) ABSTRACT

The present invention relates to a composition made from extracts of ginger (Zingiber officinale), clove (Syzygium aromaticum), green tea (Camellia sinensis), pot marigold (Calendula officinalis), broccoli (Brassica oleracea italica) and garlic (Allium sativum), suitable for use in the prevention and treatment of HPV and sexually transmitted diseases (STD). The composition increases Döderlein's bacilli, as well as additionally facilitating orgasm in women.

### OBJECT OF THE INVENTION

Composition that has a mix of extracts of the following plants: Ginger (Zingiber officinale), Clove (Syzygium aromaticum), Green Tea (Camellia sinensis), Pot marigold (Calendula officinalis), Broccoli (Brassica oleracea italica) and Garlic (Allium sativum), that provides an efficient natural prevention of daily use by increasing Döderlein bacilli to cure sexually transmitted diseases (STD) and of human papilloma virus (HPV) with additional effect of sexuality stimulant.

### BACKGROUND

a) It is known the patent BRPI1101039, topical otological pharmaceutical composition for pruritus prevention with at least one active ingredient of plant that can be chosen from different extracts, makes reference to Camellia sinensis, Brassica oleracea, Calendula officinalis, Allium cepa and many others extracts and oils, its use is topic as pruritus treatment and chronic inflammation of the skin and the auditory channel in humans and all the animals.

It has the disadvantage of separately use the active ingredients of plants. The advantage of the Composition of extracts of plants of the present inventions is that the extracts are one single composition to maintain the synergy of the effect related with the increase of Döderlein bacilli, that is applied to cure sexually transmitted diseases (STD) and early stage of human papilloma virus (HPV). Another additional effect of the composition is that it has a second use as sexually stimulant by favor female orgasm and its application is in external genitalia, vulva and vagina.

b) It is known a patent with number HU199902055A, a composition containing extracts of plants, for example Echinacea used for the treatment of bacteria and viruses, especially simple herpes, as local treatment in animals or in human. It makes reference to two plants allium and pot marigold.

This composition has the disadvantage of using basically plants of the Echinacea family in its composition. The difference of the composition of the present invention is that the composition comprises six different plants to cure STD and maintain the balance of the vaginal canal by natural defenses by increasing the Döderlein bacilli, favoring the female sexuality. The composition of the present invention is different in use, the route of administration, the mechanism and in its components.

c) It is known a publication which title is “Effect of Calendula officinalis in the treatment of the bacterial vaginosis” of the Metropolitan Autonomous University (UAM) Iztapalapa, México, thesis of December, 2002, wherein it was found that the Calendula officinalis decreases the number of bacteria after its administration. The study suggests that the Calendula officinalis has enough antibacterial activity to treat the disease, activity seen by Dumenil G. in 1980.

The advantage of the composition of the present invention is that the pot marigold is an ingredient of the composition to cure STD caused by bacteria, involving as well in the composition solving health problems by viruses, bacteria, protozoa and fungi.

d) It is known the publication in the Industrial Property Gazette, exemplary 1524 dated September, 2013, the patent MX 313127 B, formulations with content of Echinacea angustifolia and Zingiber officinale that are useful to reduce inflammation and peripheral pain.

The main difference with the composition of the present invention is the difference in its use and application of Zingiber officinale, since it is part of the composition of extracts against STD infections and initial stages of HPV injury. In the present invention Zingiber officinale is used for aphrodisiac effect to favor female orgasm, so the use and application of Zingiber officinale is different.

e) A publication “Incidence of the Döderlein bacillus and its influence on the presence of other microorganisms in the vaginal canal” is known. This work is the result of research carried out in the Cell Biology Laboratory of the Faculty of Medicine and the Mycoplasma Laboratory of the CICM-ICUAP, of the Benemérita Autonomous University of Puebla, Puebla, Mexico, in March 2008. In said publication it is said that the imbalance in the amount of bacilli of Döderlein or Lactobacillus acidophilus, during the fertile age causes an alteration in the vaginal ecosystem and, as a consequence, predisposition to infections. The statistical analysis of the investigation, regarding the amount of Döderlein bacilli present with other microorganisms showed that there is a significant association (p <0.05), that is, that the absence of the Döderlein bacilli favors the propagation of other potentially pathogenic microorganisms, concluding that the absence of a primordial component of the normal vaginal flora (Döderlein bacillus), it has a higher incidence of potentially pathogenic microbial aggregates, compared to the lower incidence of microbial aggregates in the presence of Döderlein bacilli.

This research is related because it serves as support to the composition of plant extracts of the present invention, since by increasing the Döderlein bacilli, it helps to reduce the incidence of pathogenic aggregates in STDs in general, and health of the vagina will be influenced by the increase of the Döderlein bacilli, which is achieved with the use of the present composition.

### DETAILED DESCRIPTION OF THE INVENTION

It is related to the composition made with a mixture of extracts of the following plants: Ginger (Zingiber officinale), Clove (Syzygium aromaticum), Green Tea (Camellia sinensis), Pot marigold (Calendula officinalis), Broccoli (Brassica oleracea italica) and Garlic (Allium sativum).

The selection of the plants for the composition was made using the Theory of the 5 Elements as described below:

Within the Theory of the Five Elements are two cycles: one that generates which follows the clockwise direction. SEE FIG. 3 GENERATION CYCLE; another that dominates, as the arrows mark inside the Pentagon. SEE FIG. 4 DOMAIN CYCLE.

The vertexes involved in the STD solution are: the affected vertex (V1), V2 and V3, that is, they are the immediate 2 to the vertex chosen in the anti-clockwise direction starting from the vertex that involves the origin or affected vertex problem V1. SEE FIG. 5 INVOLVED VERTEXES V1, V2 and V3.

In the vertexes of the pentagon elements, organs, plants and other functions are located.

In the present study, to resolve STDs and initial stages of HPV, we intend to give solution to the vertex V1, that is to say, an approach is made to the vertex WATER where the Kidney-Bladder pair is that by its physical location in the human body and its physiology of the adrenal glands, are associated with genitals (within the theory of the 5 elements) object of our study, which is to solve genital problems by ETS. SEE FIG. 6 LOCATION OF ORGANS AND VERTEX TO SOLVE.

The above derives in that the composition of plant extracts is useful to solve the vertex V1 or WATER with direct help of plants that must belong to vertex EARTH, METAL and WATER, because:

The Earth dominates the Water, the Metal generates the Water and the vertex itself Water.

These 3 vertexes are related to each other and the ideal to solve a physiological problem is to find plants of the affected vertex V1 or Water, plants of a vertex with generation regulation V2 or Metal and plants of another vertex that has domain V3 or Earth on V1.

A list of plants that can be selected to treat STDs and HPV, and increase Döderlein bacilli in an effective manner is presented. From the list you can choose plants that mobilize blood circulation to have the aphrodisiac effect and combine Healing-Prevention-Sexuality; they are located by vertexes V1, V2, and V3 according to the influence on the organs to be treated (KIDNEY-BLADDER).

In the three vertexes there are groups of plants, which have the capacity to influence in increasing Döderlein bacilli and cure STD. In these groups of plants you can select some of the vertex Water V1 , using other plants as support using plants of V2 and V3, previous analysis of the curative properties of each chosen plant.

TABLE 1
VERTEXES - PLANTS RELATION

| PLANTS V1 | PLANTS V2 | PLANTS V3 |
|---|---|---|
| WATER | METAL | EARTH |
| CLOVE | GINGER | GARLIC |
| MALLOW | CINNAMON | POT MARIGOLD |
| ALISMA | MINT | BROCCOLI |
| ACONITE | ABSINTHE | GREEN TEA |
| PLANTAIN | MARJORAM | GENTIAN |
| ESCHISANDRA | ONION | SOY |
| REHMANIA | TANGERINE | RHUBARB |
| ESCROFULARIA | PEPPER | GINSENG |
| PHILODENDRON | EFEDRA | SWEET POTATO |
| SOFORA | ANGELICA | CHESTNUTS |
| SARSAPARILLA | MAGNOLIA | ECHINACEA |
| FENNEL | BLACKBERRY | MELISSA |
| GINKO | CHRYSANTEMUM | CARDAMOM |
| WILD ROSE | CIMIFUGA | FERN |
| RASPBERRY | HORSETAIL | MAGNOLIA |
| | SAGE | CHAMOMILE |
| | LAUREL | CYPRESS |
| | SAGEBRUSH | CARNATION |
| | CORIANDER | AZUFAIFA |
| | SAGE | |
| | EPAZOTE | |

In this particular case the herbal composition that was chosen to give an effective solution to STDs and initial stages of HPV with its additional effect on sexuality, was by the evaluation of the action and study of the plants, relating the physiology with the organs and vertexes involved to increase the Döderlein bacilli. The increase of Döderlein bacilli provides protection by two mechanisms, one of which is to produce hydrogen peroxide and the second is to maintain an acidic environment, thus suppressing the growth of other harmful microorganisms involved in STDs.

The chosen composition of plants and their location in the vertexes is: Plants of the own vertex to solve, called vertex (V1) or Water, Clove (Syzygium aromaticum); Plants of the vertex (V2) or Metal, Ginger (Zingiber officinale); Plants of the vertex V3 or Earth, Green Tea (Camellia sinensis), Pot marigold (Calendula officinalis), Broccoli (Brassica oleracea italica) and Garlic (Allium sativum). SEE FIG. 7 LOCATION OF THE COMPOSITION IN THE VERTEXES.

Any of the plants listed in these vertexes can be used, however it was decided to elaborate the composition of extracts of Ginger (Zingiber officinale), Clove (Syzygium aromaticum), Green Tea (Camellia sinensis), Pot marigold (Calendula officinalis), Broccoli (Brassica oleracea italica) and Garlic (Allium sativum).

Thus, a composition of the area of Biotechnology was obtained, which solves problems of genital health in women by increasing the Döderlein bacilli.

The invention consists in the development of a composition of extracts of Ginger (Zingiber officinale), Clove (Syzygium aromaticum), Green Tea (Camellia sinensis), Calendula (Calendula officinalis), Broccoli (Brassica oleracea italica) and Garlic (Allium sativum) which combination results in a final composition of 3% to 8% extracts for topical use on vulva, external use, use on vaginal mucosa and that works as an effective preventive and corrective for Sexually Transmitted Diseases (STDs) and initial HPV lesions, but it also has the function of contributing to the achievement of a climax in the couple's relationship.

In the composition a pH of 4.5 is maintained, compatible with the vaginal pH.

The ingredients of the composition serve as preservatives of the same mixture of extracts.

The antifungal and antibacterial topical effect of Garlic extract was studied separately, elaborating ovules with 0.0%, 1.0% and 2.0% garlic oil with base for ova as excipient, with nipagin and nipasol as preservatives, ascorbic acid as antioxidant. The presentation in the form of ovules with 0.0%, 1.0% and 2.0% garlic oil for easy application in women incorporated into the study, indicating vaginal application with three doses, one ovule every 24 hours at bedtime, in sexually active women aged 25 to 50 who presented vaginitis and agreed to join the study, excluding women who were under treatment with antibiotics and/or systemic antifungal agents in two weeks prior to their incorporation into the study; women who used antibacterial and topical antifungals in the last 7 days were also excluded.

Samples were taken for vaginal exudate prior to the application of garlic oil ovules at 0.0%, 1% and 2% and another vaginal exudate 8 days after finishing the application of the ovules. It was indicated to apply the 3 ovules, one every 24 hours before or after their menstruation, as well as to suspend the application of them in case of irritation; it was requested to keep track of the sexual relations carried out during the application of the ovules. The results of the vaginal exudates were compared and it was observed that the garlic has antibacterial and antifungal effect with the vaginal topical use, since the report of the chemical laboratory showed a decrease of bacteria in 73% of patients incorporated to the study, and disappearance of yeasts in 88% of patients studied.

One finding was the increase in Döderlein bacilli. The increase of Döderlein bacilli is a mechanism of action discovered in the garlic extract to support the antibacterial and antifungal action of garlic in STD. The amount of Döderlein bacilli reported by the chemical laboratory after the application of the ovules is minimally doubled, as for example reported results of few Döderlein bacilli in the first exudate, in the second exudate the report is abundant Döderlein bacilli. Because of the smell of garlic it was decided to use it in a composition where other plants make synergy to fight STD. This first study of the antibacterial and antifungal effect of garlic oil, served to select the range between 1.0% and 1.8% of garlic with higher activity to increase Döderlein bacilli and the greater antibacterial effect without causing desquamation of epithelial cells by irritation.

It is decided to use 4% of extracts in the composition of the present invention which includes 1% garlic extract, as shown in the following Table 2: STUDIED PERCENTAGES OF THE COMPOSITION

| EXTRACT | 3% | 4% | 8% |
|---|---|---|---|
| Ginger (Zingiber officinale) | 0.37 | 0.37 | 0.74 |
| Green Tea (Camellia sinensis) | 0.73 | 0.73 | 1.80 |
| Pot marigold (Caléndula officinalis) | 0.90 | 0.90 | 1.80 |
| Broccoli (Brassica oleracea italica) | 0.90 | 0.90 | 1.80 |
| Clove (Syzygium aromaticum) | 0.10 | 0.10 | 0.20 |
| Garlic (Allium sativum) | 0.0 | 1.0 | 2.0 |

It was tested with percentages of the composition, varying the garlic extract, finding better results to combat STDs and early stages of HPV using the composition at 4% extracts.

The composition formula at 4% was used in a retrospective longitudinal study, in 450 patients from 18 to 60 years of age, which was performed in a Papanicolaou intake center, where an additional sample was taken for vaginal exudate, which serves as a reference before using the composition. See FIG. 1 REFERENCE GRAPH BEFORE ADMINISTERING THE COMPOSITION

As can be seen in said graph, the first three columns indicate that, of the 450 patients studied, 76% (342) were without Döderlein bacilli, while 16% (72) had them in quantity considered normal. And only 8% (36) of these patients had them with moderate excess.

The second group of three columns refers to the bacteria found before using the composition in the studied patients, concluding that 310 patients, representing 69% had a high presence of bacteria, while 27% (122 patients) had them in a moderate amount and only 4% of patients (18) were devoid of bacteria.

With respect to the third group of columns, these refer to the presence of white blood cells (Leukocytes), initial sample, in the patients who participated in the study, finding that there was a very high presence of leukocytes in 52% (234) of the 450 patients, while 31% (140 patients) had them in high form and another 17% (76) showed a moderate presence of leukocytes. With three applications of the composition, results were obtained for the Sexually Transmitted Disease (STD) solution in general by viruses, bacteria and fungi, and the first applications were initiated to solve low-grade injuries caused by the Human Papilloma Virus (HPV) in women.

The study was conducted in a period from 2004 to 2012, giving sample for three applications of the composition with herbal extracts to 4%, indicating that can be applied at the time of intercourse and requesting to return for second vaginal exudate intake at the end of the use of the composition.

With the study of the herbal composition, it was possible to detect that the composition increases the Döderlein bacilli in 83% of treated patients and in 17% of patients the initial value of Döderlein bacilli was maintained before the application as well as after the use of the composition. See FIG. 2 AFTER THE USE OF THE COMPOSITION.

Thus, we can see that, in that graph, the first block of columns refers to the Döderlein bacilli, and shows that 83% of the 450 patients treated (374) increased the presence of Döderlein bacilli, while 76 patients, which represent 17% were maintained with the amount of Döderlein bacilli without modification.

The second block of columns refers to the bacteria, and the results show that 88% (396 patients) had bacteria decrease with the use of the composition, while 12% (54 patients) remained unchanged.

Finally, the third block of columns shows the modifications obtained in the amount of leukocytes, with the use of the composition, finding that 441 patients, representing 98% of the 450 studied, returned to reference values, considered adequate in the exudate vaginal and only 2% (9 patients) remained without changes in the amount of leukocytes.

A decrease in bacteria was detected in 88% of the population studied, as well as a decrease in leukocytosis in 98% of patients studied for vaginal exudate. SEE FIG. 2 AFTER THE USE OF THE COMPOSITION.

Another characteristic obtained with the composition of extracts is the obtaining of a local aphrodisiac effect in women, favored by the use in the composition of spicy plants that favor blood circulation in situ, which generate local heat and sensitize the nerve fibers of muscles of the vulva, the spicy plants that form the composition are A, B and F mainly of the composition studied, A.—Ginger (Zingiber officinale), B.—Clove (Syzygium aromaticum) and F.—Garlic (Allium sativum) being these responsible for the stimulating effect to favor sexual pleasure in women. This aphrodisiac sensitizing effect of the nerve fibers in the vulva is referred to with the data collected by the survey and the use of the composition at the time of the sexual relationship.

The questions of the applied questionnaire that refer to favoring the achievement of the female orgasm are: 5.—What effects did it obtain during its use? and 6.—Which of these effects did you like the most? where the most frequent expressions that the patients manifested were:
Orgasm
Stimulant
Local heat
Longer duration of sexual intercourse
Flavoring
It does not itch
Lubricant
Absence of pain
Freshness
No unpleasant smell after the relationship
Did not end up irritated
I did not know that this exists

To assess that increases genital sensitivity, increases freshness, local heat and generates a slight tingling, thus facilitating the achievement of female orgasm, referred even by women who had never had orgasm, the following questionnaire was applied:

### EXAMPLES

For the purpose of assessing the effects of the extracts, the composition was presented to patients in samples as a creamy product that complies with a cosmetic form of easy application, which was suggested to be applied to external genitalia, vulva and/or penis, during sexual intercourse.

Three samples were given to apply three times. Benefits were evaluated in sexuality, after the use of the composition and the interview when applying the questionnaire.

For the case of 76 patients, which represent 17% of the total patients in the study, who maintained the amount of Döderlein bacilli without modification, the number of bacteria decreased.

In the population of women incorporated into the study for the use of the composition, 12% were reported with yeasts before the application disappearing in the second intake, which serves as a parameter to say that the composition solves STD by fungi, candida albicans.

In the questionnaire applied after using the composition, 97% of patients answered that they would like to use an herbal product to: Cure—prevent diseases and enjoy sexuality.

The 100% of the surveyed population is interested in knowing and receiving a sample of herbal product to increase sexual satisfaction with their partner.

Of the surveyed population, 100% felt good about using the composition to assess the favorable effect of orgasm and rated the use of the composition as very satisfactory.

With the questions: What effects did you get during your use? And which of these effects did you like the most? The answers were very varied for the effects, so it is determined that it increases genital sensitivity, increases freshness, local heat and generates a slight tingling, thus facilitating the achievement of female orgasm, referred even by women who had never had orgasm, as they expressed I did not know that this exists!, for sexual pleasure.

The hot and spicy nature of ginger, garlic and clove are the main contributors to this aphrodisiac effect because they mobilize blood circulation at the contact site, generate heat and increase turgor and sensitivity in external genitalia causing sexual pleasure.

In the study carried out with the herbal composition men were included but 100% referred changes only as a lubricant and that women are excited.

The reports of vaginal exudates before using the composition and after its use were made by a chemical laboratory that analyzed the samples of fresh vaginal exudate and Gram stain.

The chemical laboratory pointed out that when they report yeast in the samples studied, it is referred to the existence of vaginal candidiasis; STD caused by the Candida Albicans Fungus.

The chemical laboratory also reported, as scarce, moderate and/or elevated an altered parameter, which is equivalent to reporting crosses +, ++ and +++

The composition was applied in injuries of the lips, observing that it stops the inflammatory process, decreases the pain and accelerates the healing of injuries produced by labial and buccal Herpes virus.

To evaluate the initial treatment stages for HPV, the herbal composition was used by applying it directly to the site of the injury, that is, it was applied to the cervix as cauterization with the composition to observe the re-establishment of the epithelia, checking for a new take of the Vaginal Cytology due to the disappearance of injuries and ulcers when reporting healthy tissue.

Another application scheme in patients that resulted with low grade intraepithelial injuries consisted in the use of the composition for 7 more days, and in this case the reports were kept at IMSS (Mexican Institute of Social Security) clinic to which the patients belonged, obtaining only one verbal report referred by his treating doctor who informed them that they were already well in the new vaginal cytology, from which it is deduced that the composition provides an acceleration in the regeneration of the epithelium when it is a low grade injury.

The above reaffirms that the composition of plant extracts increases the Döderlein bacilli, important for the treatment of Sexually Transmitted Diseases (STDs) and early stages of the appearance of the Human Papilloma Virus (HPV) with an additional aphrodisiac effect.

### BRIEF DESCRIPTION OF THE FIGURES

FIG. 1 REFERENCE GRAPH BEFORE APPLYING THE COMPOSITION. This graph shows the data of three elements that were relevant when carrying out the clinical analyzes before applying the composition (Vaginal Exudate first shot) of the patients incorporated in the study; specifically, it is the presence of Döderlein Bacilli, Bacteria and Leukocytes, the three parameters on the abscissa axis, against the percentage of patients studied, axis of the ordinates.

FIG. 2 AFTER THE USE OF THE COMPOSITION. The second graph shows the result obtained with the application of the composition to the patients under study, basically as regards the modification of the presence of the elements considered relevant in the clinical analyzes carried out at the beginning of the study.

FIG. 3 GENERATION CYCLE. It deals with the location of the GENERATION CYCLE in the Theory of the Five Elements which follows the clockwise direction, marked by the continuity of the arrows in the periphery of the pentagon.

FIG. 4 DOMAIN CYCLE. In this figure are located arrows inside the pentagon, which make connotation to the CYCLE OF DOMAIN, expressing that the Earth dominates the Water, the Water dominates the Fire, the Fire dominates the Metal, the Metal dominates Wood and this in turn dominates to Earth, closing the cycle.

FIG. 5 VERTEXES INVOLVED V1, V2 y V3. It is the representation of the involved vertexes to solve the ETS and HPV, determining as V1 vertex or vertex Water and two vertexes plus V2 and V3 to correct the affected vertex V1: in this particular case correct ETS and HPV. In this figure we indicate the affected vertex (V1), V2 and V3, involved for the best response, that is, they are 2 immediate to the vertex chosen in the anti-clockwise direction starting from the vertex that involves the origin problem V1.

FIG. 6 LOCATION OF ORGANS AND VERTEX TO SOLVE. It refers to the location of elements, organs and other functions, in the vertexes of the pentagon. In this graph, the pair of organs KIDNEY-BLADDER (KB) is indicated to give solution to V1, an approach is made to the water vertex where the Kidney-Bladder pair is that by its physical location in the human body and its physiology of the Adrenal glands is associated with genitalia object of our study, which is to solve genital problems by STD.

FIG. 7 LOCATION OF THE COMPOSITION IN THE VERTEXES. It is the representation of the composition of plant extracts related to the vertex to which they belong, involving vertex Earth V3, Metal V2 and water V1.

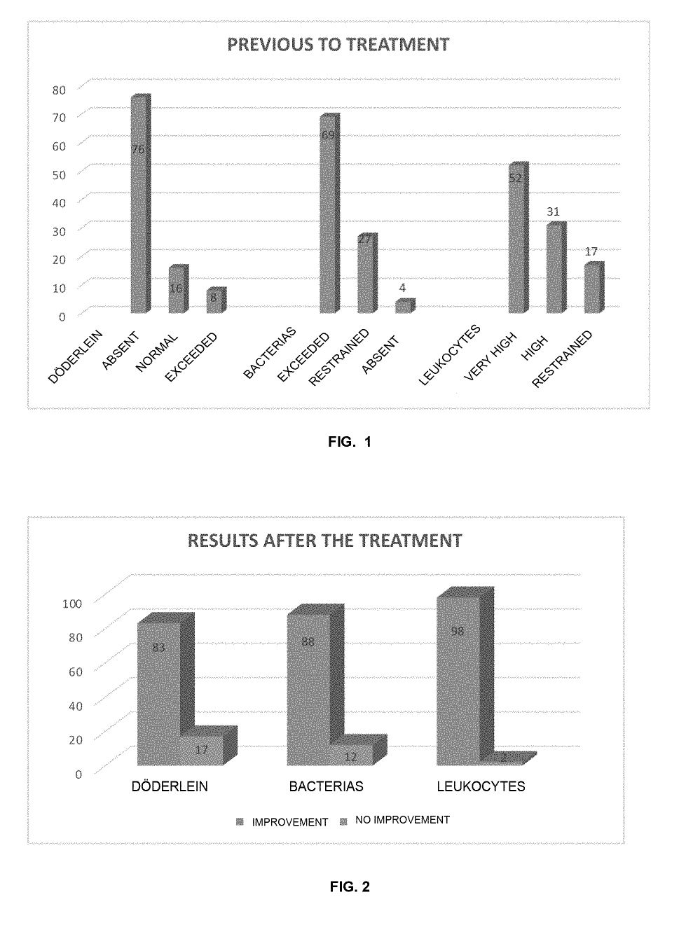

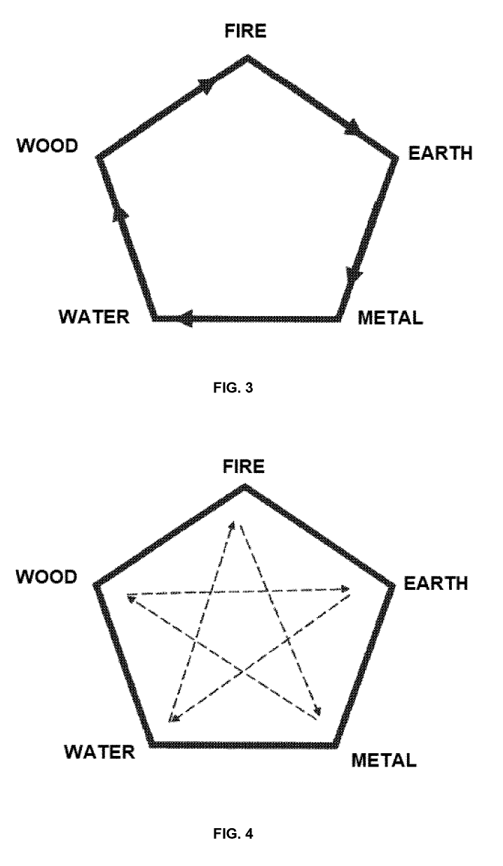

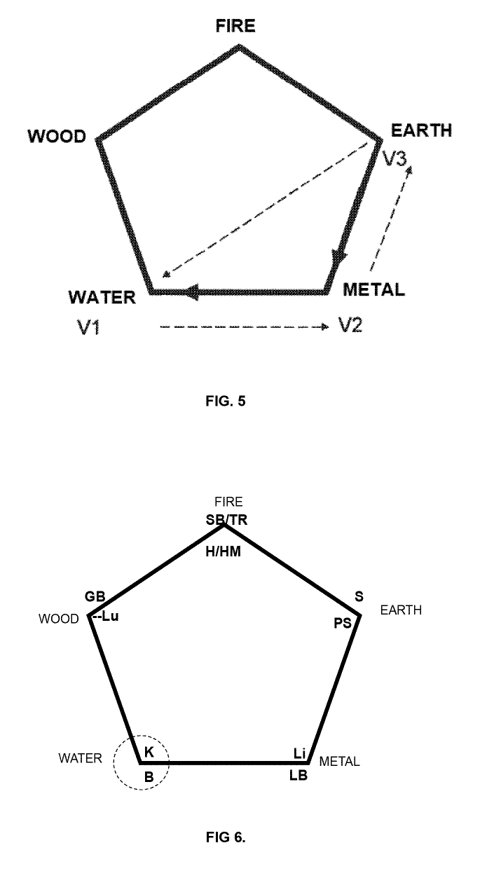

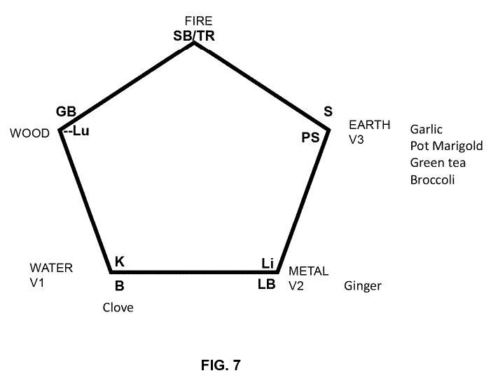

1. A composition of plant extracts comprising the components:
(a) ginger (Zingiber officinale),
(b) clove (Syzygium aromaticum),
(c) green tea (Camellia sinensis),
(d) pot marigold (Calendula officinalis),
(e) broccoli (Brassica oleracea italica),
(f) garlic (Allium sativum).

2. The composition according to claim 1, wherein the total of the components represents from 4% to 8% of the composition as a whole.

3. The composition according to claim 1, wherein the total of the components are present in the following proportions with respect to the composition as a whole:
(a) ginger (Zingiber officinale) 0.37% to 0.74%,
(b) clove (Syzygium aromaticum) 0.10% to 0.20%,
(c) green tea (Camellia sinensis) 0.73% to 1.46%,
(d) pot marigold (Calendula officinalis) 0.90% to 1.80%,
(e) broccoli (Brassica oleracea italica) 0.90% to 1.80%,
(f) garlic (Allium sativum) 1.0% to 2.0%.

4. The composition according to claim 3, wherein the preferred proportion of garlic is between 1.0% and 1.8% with respect to the composition as a whole.

5. The composition according to claim 1, wherein the composition comprises an excipient that is present in an amount of 92% to 96% of the composition as a whole.

6. The composition according to claim 1, for use in the prevention and/or treatment of sexually transmitted diseases (STD) by increasing the Döderlein bacilli.

7. The composition according to claim 6, wherein the composition presents an antimicotic, antimicrobial, and/or antiviral activity.

8. The composition of claim 1, for use as an aphrodisiac favoring female orgasm.

9. The composition of claim 1, wherein the composition is administrable via topical.

10. The composition according to claim 9, wherein the composition is suitable to be in the form of cream, gel, compresses, ointment, powder envelopes for showers, ovules, or syringes for vaginal application.

11. The composition of claim 9, wherein the composition is administrable to the external genitals and/or vaginal mucosa.
